(19) United States
(12) Patent Application Publication
(10) Pub. No.: US 2025/0040850 A1
(43) Pub. Date: Feb. 6, 2025

(54) SENSOR INCLUDING ELECTRICALLY CONDUCTIVE ABRASIVE MATERIAL

(71) Applicant: COVIDIEN LP, Mansfield, MA (US)

(72) Inventors: Denis Glozman, Modiin (IL); Shai Fleischer, Modiin (IL); Ilan Breskin, Tel Aviv-Yafo (IL); Alon Sasson, Rehovot (IL)

(21) Appl. No.: 18/751,971

(22) Filed: Jun. 24, 2024

Related U.S. Application Data
(60) Provisional application No. 63/517,791, filed on Aug. 4, 2023.

(51) Int. Cl. A61B 5/259; A61B 5/266; A61B 5/28; A61B 5/291

(52) U.S. Cl. CPC A61B 5/259; A61B 5/266; A61B 5/28; A61B 5/291; A61B 2562/0217; A61B 2562/14

(57) ABSTRACT

An example sensor includes an electrode assembly including an electrode well and an electrically conductive material disposed within the electrode well, the electrically conductive material comprising abrasive particles. The abrasive particles are configured to displace at least a portion of a layer on or of skin of a patient upon application of the sensor to the skin of the patient.

### CROSS-REFERENCE TO RELATED APPLICATION

This application claims priority to and the benefit of U.S. Provisional Patent Application No. 63/517,791, entitled “SENSOR INCLUDING ELECTRICALLY CONDUCTIVE ABRASIVE MATERIAL,” filed on Aug. 4, 2023, which is incorporated herein by reference in its entirety for all purposes.

### TECHNICAL FIELD

The disclosure relates to medical sensors including electrodes.

### BACKGROUND

Some medical monitors are configured to noninvasively monitor one or more physiological parameters of a patient using external electrodes. For example, a bispectral index (BIS) brain monitoring system is configured to monitor brain activity of a patient based on bioelectrical brain signals sensed via external electrodes (e.g., via an electroencephalogram (EEG)). The external electrodes can be applied to various anatomies of the patient (e.g., the temple and/or forehead). For example, some sensors for BIS monitoring include a single strip that includes several electrodes for placement on the forehead to noninvasively acquire an EEG signal.

### SUMMARY

The present disclosure describes sensors including one or more electrodes configured to monitor one or more physiological parameters of a patient, e.g., cardiac signals, brain signals, and the like, as well as devices, systems, and techniques related to the sensors. The sensors include one or more electrodes configured to noninvasively sense a physiological parameter of a patient via electrical contact with the patient (e.g., electrical contact with skin of the patient) and an electrically/ionically conductive material that is configured to improve electrical/ionic conductivity between the one or more electrodes and the patient and reduce the impedance of the electrode-to-patient connection. For example, the electrically/ionically conductive material may include an electrically/ionically conductive electrolytic gel configured to be positioned between skin of a patient and an electrode, e.g., in an electrode well.

The electrically/ionically conductive material comprises abrasive particles configured to displace at least a portion of a layer on or of the skin of the patient (the layer having a relatively high impedance) when the sensor is applied to the skin to help reduce the impedance of an electrical path between the patient (e.g., the epidermis) and the electrode. For example, an outermost layer of the epidermis of the patient may include dead skin cells and/or body oils, which may have a higher electrical impedance than a more inner layer of skin of the patient, and the abrasive particles may displace this first layer of the epidermis to expose a more electrically conductive layer of skin. In this way, the abrasive particles can help reduce the electrical impedance between the skin of the patient and the electrode of the sensor. Displacing a portion of a layer on the skin can also help improve wetting of the conductive material directly to the skin of the patient.

Herein, the electrically/ionically conductive material may be referred to as just an “electrically conductive” material, although it is to be understood that the electrically conductive material may be an electrically and/or ionically conductive material). In examples disclosed herein, a sensor includes an electrically conductive material (e.g., an electrically/ionically conductive gel or electrolytic material or gel) comprising abrasive particles. For example, the sensor may comprise a backing layer, an electrode disposed on the backing layer, a foam layer disposed on at least a portion of the backing layer, and an adhesive disposed on an outer surface of the foam layer and configured to adhere the sensor to a patient. The backing layer, foam layer, and gel may form an electrode well, and the electrically conductive material may be disposed within the electrode well, e.g., within a sponge configured to at least partially retain the electrically conductive material. The sensor may be at least partially deformable, such than when the sensor is applied to the patient, e.g., the adhesive is pressed to the skin of the patient, a force is applied to the sensor in a direction towards the surface of the skin causing the electrically conductive material and abrasive particles to flow within the electrode well and along the patient's skin such that the abrasive particles displace at least a portion of a layer on or of the skin and causes the electrically conductive material to wet to the skin.

The sensors and techniques disclosed herein include a number of benefits and advantages. For example, including an electrically conductive material comprising abrasive particles with the sensor may eliminate the need for a separate skin preparation step to remove the electrically insulating layer before applying the sensor to the patient. Eliminating a separate skin preparation step may improve adhesion of the adhesive to the patient by removing the need to prepare the patient's skin with a separate abrasive gel that is otherwise not contained within the electrode well and may extend to areas on which the adhesive is to be applied, and which may interfere with the adhesion of the sensor to the skin of the patient. In some examples, including an electrically conductive material comprising abrasive particles with the sensor also eliminates the need for other sensor components, such as tines configured to displace the layer on the skin.

In some embodiments, this disclosure describes a sensor including: an electrode assembly including an electrode well; and an electrically conductive material disposed within the electrode well and comprising abrasive particles, wherein the abrasive particles are configured to displace at least a portion of a layer on or of skin of a patient upon application of the sensor to the skin of the patient.

In some embodiments, this disclosure describes an assembly including: a sensor including: an electrode assembly including an electrode well, the electrode assembly including: a backing layer; an electrode disposed on the backing layer; a foam layer disposed on at least a portion of the backing layer; an adhesive disposed on at least a portion of the foam layer and configured to adhere the sensor to a patient, wherein the electrode well is defined by the foam layer and the backing layer; and an electrically conductive material disposed within the electrode well and comprising abrasive particles, wherein the abrasive particles are configured to displace at least a portion of a layer on or of a skin of a patient upon application of the sensor to the skin of the patient; and a peelable layer configured to contain the electrically conductive material within the electrode well and configured to releasably adhere to, and cover, an outer surface of the adhesive.

In some embodiments, this disclosure describes a method including: positioning a sensor on a surface of a skin of a patient, the sensor including an electrode assembly including an electrode well; and an electrically conductive material comprising abrasive particles; applying a force to the sensor to adhere the adhesive to the surface about a perimeter of the electrode well thereby sealing the electrode well; and applying a force to the sensor in a direction towards the surface of the skin, wherein the application of the force causes the abrasive particles to displace at least a portion of a layer on or of the skin of the patient.

The details of one or more examples are set forth in the accompanying drawings and the description below. Other features, objects, and advantages will be apparent from the description and drawings, and from the claims.

### BRIEF DESCRIPTION OF THE DRAWINGS

Advantages of the disclosed techniques may become apparent upon reading the following detailed description and upon reference to the drawings in which:

FIG. 1 is a conceptual block diagram illustrating an embodiment of a monitoring system configured to be used with a sensor;

FIG. 2A is a perspective view of an embodiments of a sensor including an electrically conductive material including abrasive particles;

FIG. 2B is an exploded perspective view of the sensor of FIG. 2A;

FIG. 3 is cross-sectional view of the sensor of FIG. 2A, the cross-section being taken along the line A-A′ illustrated in FIG. 2A;

FIG. 4 is a flow diagram of an embodiment of a method of using a sensor including an electrically conductive material including abrasive particles;

FIG. 5 is cross-sectional view of the sensor of FIG. 2A before being applied to a patient, the cross-section being taken along the line A-A′ illustrated in FIG. 2A;

FIG. 6 is cross-sectional view of the sensor of FIG. 2A applied to a patient and before the electrode well is pressed, the cross-section being taken along the line A-A′ illustrated in FIG. 2A;

FIG. 7A is cross-sectional view of the sensor of FIG. 2A applied to a patient with a force applied to the sensor to press the electrode well, the cross-section being taken along the line A-A′ illustrated in FIG. 2A;

FIG. 7B is cross-sectional view of the sensor of FIG. 2A applied to a patient and after the electrode well is pressed, the cross-section being taken along the line A-A′ illustrated in FIG. 2A; and

FIG. 8 is a table illustrating a comparison of the impedances of several sensors.

### DETAILED DESCRIPTION

In examples disclosed herein, a sensor includes an electrically conductive material comprising abrasive particles configured to displace at least a portion of a layer on or of a surface of the skin of the patient upon application of the sensor to the skin of the patient. The layer can include, for example, at least part of an outermost layer of an epidermis of a patient (e.g., the stratum corneum), which can include dead skin cells, body oils, dust, debris, or the like that can have a higher impedance than a more inner layer of the skin or otherwise interfere with sensing via one or more electrodes. The abrasive particles may displace this outermost layer of the epidermis to expose a more electrically conductive layer of skin, which helps reduce the impedance between one or more electrodes of the sensor and skin of the patient. The electrically conductive material (e.g., electrolytic gel) improves electrically conductivity between the one or more electrodes and the skin of the patient, such as by reducing an impedance between the one or more electrodes and the skin of the patient. For example, an electrolytic gel may wet to the skin and to an electrode, wet between the skin and the electrode, or otherwise increase a surface area of contact between the skin and the one or more electrodes of the sensor.

The sensor may be adhered to the skin of the patient and pressed towards the skin of the patient to engage the abrasive particles with the skin and improve electrical conductivity between an electrode and the skin. In some examples, the sensor includes an electrode well including an electrode and the electrically conductive material. The electrode well is configured to be compressible such that upon applying a force to sensor (e.g., pressing the sensor), the electrode well compresses and cause the electrically conductive material to move or flow within the electrode well. The electrode well is configured to contain the electrically conductive material when the sensor is applied to the skin of the patient, and the abrasive particles of the electrically conductive material are configured to clean, disrupt, move, abrade, or otherwise displace at least a portion of a layer on the patient's skin to improve wetting of the electrically conductive material to the outer surface of the patient's skin, e.g., the outer surface of the epidermis of the patient.

In some examples, the electrically conductive material is configured to absorb at least a portion of the displaced layer on or of the skin. In some examples, the electrically conductive material is configured to hold at least a portion of the displaced layer in suspension at least for a first amount of time, e.g., the amount of time the sensor is in use, which may be minutes, hours, days, or longer. For example, in contrast with sensors utilizing skin preparation techniques that remove at least a portion of the layer on the skin (e.g., at least from an area of the skin corresponding to the footprint of the sensor when applied to the skin), the electrically conductive material and abrasive particles are configured to reduce the electrical impedance between the skin and sensor electrode(s) while the material of the displaced layer (e.g., dead skin cells, oils, dust, debris, and the like) remain in the general footprint of the sensor (e.g., and may be moved from the skin, such as by being absorbed or held in suspension for at least a portion of time within the conductive material).

The inclusion of abrasive particles in an electrically conductive material that is included with the sensor may eliminate a skin cleaning or other skin preparation step. For example, with some other techniques, a layer on or of the skin having a relatively high impedance, e.g., a layer of dead skin cells and/or body oils, is removed before applying the sensor to the skin by abrading the skin with an abrasive such as sandpaper, an abrasive pad, an abrasive wool, or some other rough surface on the skin over the area to which the sensor is to be applied. In addition or instead, a sensor includes tines configured to engage with the skin to mechanically displace a layer of the skin upon application of the sensor to the skin. The tines can be, for example, within an electrode well of the sensor. A sensor including tines may eliminate a skin preparation step, but may result in the tines pressing into the skin of the patient during use of the sensor. The tines can also increase the complexity of the sensor design, e.g., is an additional component to be manufactured and packaged within the sensor, and which may require the use of further additional components such as spacers to position the integrated component (e.g., tines) correctly relative to the skin of the patient upon application of the sensor to the skin.

The sensors described herein that include an electrically conductive material comprising abrasive particles can eliminate the need for both a separate skin preparation step (prior to applying the sensor to the patient) and sensor components, such as tines configured to displace the layer on the skin. Eliminating a separate skin preparation step may save time and improve adhesion of an adhesive of the sensor to the patient by reducing the possibility that the abrasive material or an electrically conductive material applied to skin before the sensor is positioned between the adhesive and the skin of the patient. That is, when a clinician prepares skin of a patient prior to applying the sensor to the skin, some of the skin preparation material may be left on the skin where the adhesive is to be applied and may interfere with adherence of the sensor to the skin. In contrast, the sensors described herein are configured to retain the electrically conductive material (including the abrasive particles) to reduce the possibility that the electrically conductive material will be positioned between an adhesive of the sensor and skin of the patient.

The sensors described herein may include any suitable sensor, such as, but not limited to, an EEG sensor or an electrocardiogram (ECG) sensor.

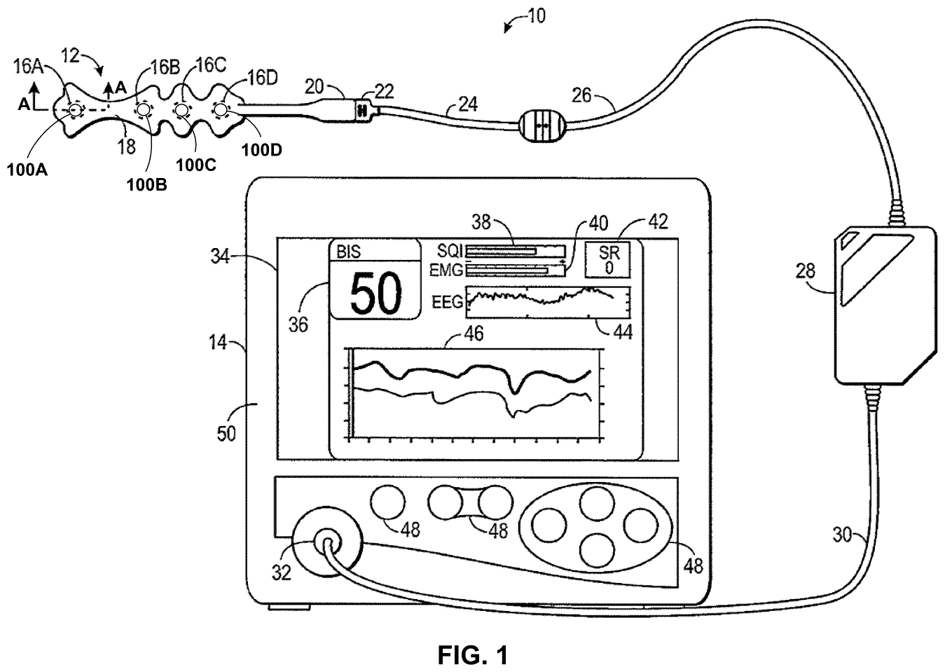

FIG. 1 is a conceptual block diagram illustrating an embodiment of a monitoring system 10. In the example shown in FIG. 1, monitoring system 10 includes sensor 12 and electroencephalogram (EEG) monitor 14. The sensor 12 includes one or more electrodes 16 (e.g., four electrodes 16A, 16B, 16C, and 16D as shown in FIG. 1, but can include one electrode, two electrodes, three electrodes, or more than four electrodes in other examples). In other examples, the monitor 14 can be configured to monitor one or more other physiological parameters of a patient instead of or in addition to EEG signals, such as, but not limited to, ECG signals. Thus, while the one or more electrodes 16 are primarily referred to herein as being configured to acquire EEG signals, in other examples, the one or more electrodes 16 can be configured to sense other physiological parameters of a patient in other examples.

The one or more electrodes 16 may have any suitable configuration. In some embodiments, the one or more electrodes 16 include a printed conductive ink supported within a flexible sensor body 18 to provide enhanced flexibility and conformance to patient tissue. In some examples, one or more of the one or more electrodes 16 may be self-adherent and self-prepping.

The sensor 12 further includes an electrically conductive material configured to increase the electrical conductivity between the one or more electrodes 16 and the patient, such as by lowering the impedance of an electrical path between the one or more electrodes 16 and the patient (e.g., skin of the patient). While the electrically conductive material is primarily referred to herein as an electrically conductive gel (or “conductive gel”), in other examples, the electrically conductive material can have any suitable configuration (e.g., viscosity). A gel may have sufficient viscosity to exhibit no flow when in the steady state (e.g., in the absence of an external force causing the gel to move) and may be particularly well suited to remain between the one or more electrodes 16 and a surface (e.g., skin of a patient).

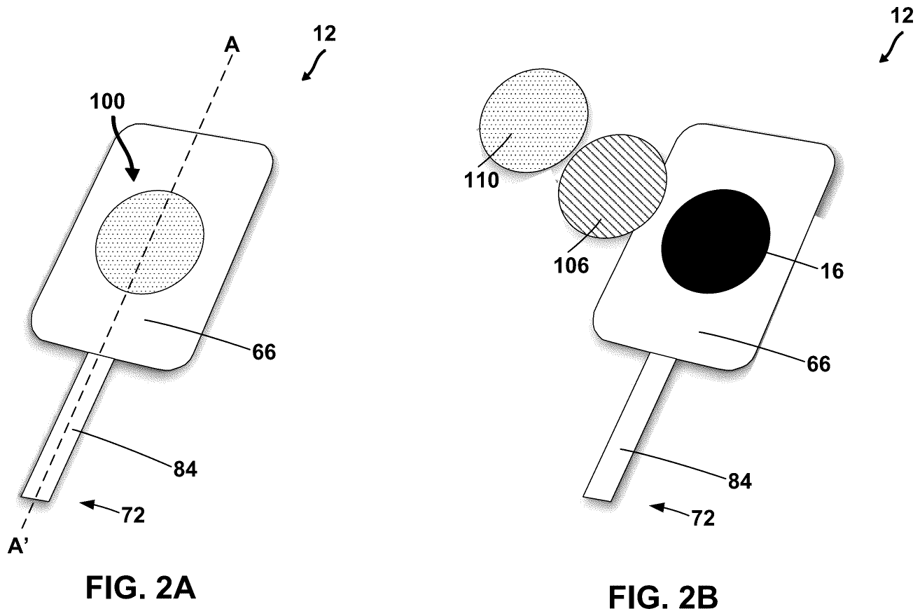

The one or more electrodes 16 may each be in, or at least partially define, an electrode well (e.g., a containment assembly), as illustrated and described further below with respect to FIGS. 2A-7. In particular, each electrode well may be a space within which conductive gel is stored. In some embodiments, at least one of the one or more electrodes 16 includes an electrode well. It should be appreciated that the electrode well may be configured to store an electrically conductive material in a form other than a gel, such as a more liquid or solid form. In the example shown in FIG. 1, the sensor 12 includes electrode wells 100A, 100B, 100C, and 100D (generally referred to as an electrode well or wells 100) corresponding to electrodes 16A, 16B, 16C, and 16D. In other embodiments, however, only a subset of the one or more electrodes 16 may each include a respective or shared electrode well 100.

The sensor 12 includes a conductive gel (or other electrically conductive material) comprising abrasive particles configured to displace a layer on or of the skin of the patient. The sensor 12 is configured such that when the sensor 12 is positioned on skin of a patient and a force (e.g., depressive force, compressive force) is applied to the sensor 12 towards the skin, the conductive gel including the abrasive particles flows and/or otherwise moves within the electrode well 100 to cause the abrasive particles to abrade and/or otherwise displace at least a portion of a layer on or of skin (e.g., relatively high impedance dead skin cells, oils, or the like). This displacement of the at least a portion of the layer on or of the skin increases the electrical conductivity of a pathway between the respective electrode 16 and the patient (e.g., patient's skin) such as by enabling the conductive gel to wet out to (e.g., couple to, electrically couple to, contact) a lower impedance layer of the patient's skin. Additionally or alternatively, depression of the sensor 12 may displace the conductive gel within the electrode well 100 and cause the conductive gel to flow towards the skin of the patient. To this end, flow of the conductive gel may increase a surface area (e.g., contact area, contact surface) of which the conductive gel wets to or contacts the skin of the patient. In some embodiments, the conductive gel may be configured to absorb and/or hold the displaced material of the layer on or of the skin in suspension, at least for a period of time.

The sensor 12 is configured to electrically connect to the monitor 14. In the example shown in FIG. 1, the sensor 12 includes a connector 20, which couples through a connector 22 to a cable 24 (e.g., a patient interface cable), which in turn may be coupled to a cable 26 (e.g., a pigtail cable). In other examples, the sensor 12 may be directly coupled to the cable 26 thereby eliminating the cable 24. The cable 26 may be coupled to a digital signal converter 28, which in turn is coupled to cable 30 (e.g., a monitor interface cable). In some examples, the digital signal converter 28 may be embedded in the monitor 14 to eliminate cables 26 and 30. The cable 26 may be coupled to the monitor 14 via a port 32 (e.g., a digital signal converter port). The sensor 12 can be electrically connected to the monitor 14 using other techniques/configurations in other examples.

In some embodiments, the monitor 14 is configured to monitor one or more physiological parameters of a patient via the sensor 12. For example, the sensor 12 may be a bispectral index (BIS) sensor 12 and the monitor 14 may be configured to monitor brain activity of the patient based on EEG signals received from the one or more electrodes 16 of the sensor 12. The monitor 14 includes processing circuitry configured to algorithmically determine a bispectral index from the EEG signals, which may indicate a level of consciousness of a patient during general anesthesia.

In the example shown in FIG. 1, the monitor 14 includes display 34 configured to display information, such as, but not limited to, sensed physiological parameters, historical trends of physiological parameters, other information about the system (e.g., instructions for placement of sensor 12 on the patient), and/or alarm indications. For example, the monitor 14 may display a BIS value 36, a signal quality index (SQI) bar graph 38, an electromyograph (EMG) bar graph 40, a suppression ratio (SR) 42, an EEG waveform 44, and/or trends 46 over a certain time period (e.g., one hour) for EEG, SR, EMG, SQL and/or other parameters. The BIS value 36 represents a dimensionless number (e.g., ranging from 0, i.e., silence, to 100, i.e., fully awake and alert) output from a multivariate discriminate analysis that quantifies the overall bispectral properties (e.g., frequency, power, and phase) of the EEG signal. The SQI bar graph 38 (e.g., ranging from 0 to 100) indicates the signal quality of the EEG channel source(s) based on impedance data, artifacts, and other variables. The EMG bar graph 40 (e.g., ranging from 30 to 55 decibels) indicates the power (e.g., in decibels) in a particular frequency range that includes power from muscle activity and other high-frequency artifacts. The SR 42 (e.g., ranging from 0 to 100 percent) represents the percentage of epochs over a given time period (e.g., the past 63 seconds) in which the EEG signal is considered suppressed (i.e., low activity). In some examples, monitor 14 may display a verification screen verifying the proper placement of each electrode 16 of sensor 12 on the patient.

Additionally, the monitor 14 may include various activation mechanisms 48 (e.g., buttons and switches) to facilitate management and operation of the monitor 14. For example, the monitor 14 may include function keys (e.g., keys with varying functions), a power switch, adjustment buttons, an alarm silence button, and so forth, which can be provided by buttons or by a touchscreen display 34.

Although the sensor is described as being used with the monitor 14 as illustrated and described with reference to FIG. 1, in other examples, the sensor 12 can be used with other types of monitors.

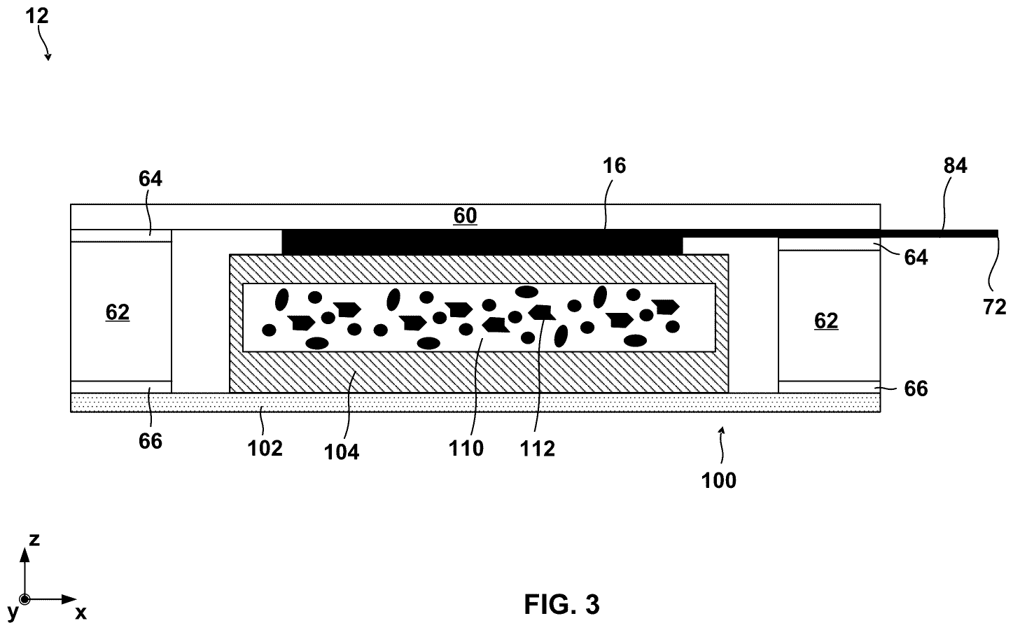

FIGS. 2A-3 illustrate various views of an embodiment of the sensor 12 including an electrically conductive material including abrasive particles, and are described together. FIG. 2A is a perspective view of the sensor 12 and FIG. 2B is an exploded perspective view of the sensor 12 of FIG. 2A. FIG. 3 is a schematic cross-sectional view of the sensor 12 of FIG. 2A, the cross-section being taken along the line A-A′ illustrated in FIG. 2A.

The sensor 12 is an embodiment of the sensor 12 as described with reference to FIG. 1. As illustrated in FIG. 2A, the sensor 12 includes an electrode well 100 disposed within (e.g., surrounded by, encased by) an area of a patient contacting adhesive 66, and a conductor 84. FIGS. 2A and 2B are views of a contacting side, or measurement side, or active area of the sensor 12, e.g., showing the surface of the sensor 12 which is to be applied to the skin of a patient. As shown in the exploded view of FIG. 2B, the electrode well 100 includes an electrode 16, a sponge 106, and electrically conductive material 110 including abrasive particles (e.g., an electrically and/or ionically conductive material, which may be an electrolytic material).

As shown in FIG. 3, the sensor 12 includes a plurality of layers in a depth direction, e.g., the z-direction as shown. The plurality of layers of the sensor 12 includes a backing layer 60, a foam layer 62, and a first adhesive 64 configured to secure the foam layer 62 to the backing layer 60. In some examples, the sensor 12 may include a patient contacting adhesive 66 configured to secure the sensor 12 to a patient, and a peelable layer 102 configured to releasably adhere to and cover (e.g., protect, block, impede) the patient contacting adhesive 66 (e.g., an outer surface of the patient contacting adhesive 66), for example, when the sensor 12 is not in use during shipping and storing of the sensor 12. The patient contacting adhesive 66 may be located on a side of the foam layer 62 opposite a side at which the first adhesive 64 is located. The backing layer 60 may be a structural layer, e.g., a base layer configured to provide structural support. The backing layer 60 may define at least a portion of the electrode well 100. The backing layer 60 may be constructed from any flexible material suitable for use in medical devices, such as, but not limited to, polyester, polyurethane, polypropylene, polyethylene, polyvinylchloride, acrylics, nitrile, polyvinylchloride (PVC) films, acetates, or similar materials that facilitate conformance of the sensor 12 to the patient. The foam layer 62 may provide padding and additional comfort to the patient. As an example, the foam layer 62 may include any foam material suitable for use in medical applications, such as, but not limited to, polyester foam, polyethylene foam, polyurethane foam, or the like.

In the example shown, the backing layer 60 of sensor 12 may be configured to facilitate retention of the sensor 12 on a patient, e.g., to maintain pressure of the corresponding electrode 16 positioned on the backing layer 60 against the patient's forehead, temple, or other external surface or skin of the patient. The electrode 16 is positioned on the backing layer 60, e.g., at the center of backing layer 60 as shown in FIG. 2B. In some embodiments, the electrode 16 may be positioned on the backing layer 60 at a non-centered location. A shape of the backing layer 60 may be reflected in the shape of the foam layer 62 and the first adhesive 64. In particular, the portions of the foam layer 62 and the first adhesive 64 may attach to corresponding portions of the backing layer 60. The foam layer 62 and the first adhesives 64 may each include a respective hole (e.g., forming the electrode well 100) that corresponds to the position of the electrode 16 (e.g., on the backing layer 60) to facilitate electrical contact with the patient.

In some embodiments, the foam layer 62, the first adhesive 64, and the patient contacting adhesive 66 may be provided as discrete layers as illustrated or may be provided as a single piece. That is, the foam layer 62, the first adhesive 64, and the patient contacting adhesive 66 may be provided as a double-coated foam layer. Any or all of the foam layer 62, the first adhesive 64, the electrode 16, and the backing layer 60 may form the electrode well 100.

The electrode 16 includes any suitable electrically conductive material. For example, the electrode 16 may be formed from flexible electrically conductive materials, such as one or more conductive inks. In some examples, the electrode 16 may be produced by printing (e.g., screen printing or flexographic printing) a conductive ink on the backing layer 60 and allowing the ink to dry and/or cure. In some examples, the ink may be thermally cured. The sensor 12 may also include a plurality of conductors 84 disposed (e.g., screen or flexographically printed) on the backing layer 60 and configured to transmit signals to and from the electrode 16 and to enhance flexibility of the sensor 12, for example, as electrical connections to the electrode 16. The plurality of conductors 84 may be formed from the same or a different conductive ink than the electrode 16.

Suitable conductive inks for the electrode 16 and/or the plurality of conductors 84 may include inks having one or more conductive materials such as metals (e.g., copper (Cu) or silver (Ag)) and/or metal ions (e.g., silver chloride (AgCl)), filler-impregnated polymers (e.g., polymers mixed with conductive fillers such as graphene, conductive nanotubes, metal particles), or any ink having a conductive material capable of providing conductivity at levels suitable for performing physiological, EEG, and/or other electrical measurements. As an example, the electrode 16 and/or the plurality of conductors 84 may be formed from an ink having a mixture of Ag and AgCl. In some examples, silver and salts thereof (e.g., Ag/AgCl) may be desirable to use for the electrode 16 and/or the plurality of conductors 84 due to its enhanced stability (e.g., compared to copper and copper salts) during certain medical procedures, such as defibrillation. For example, the Ag/AgCl may enable the sensor 12 to depolarize within a desired amount of time (e.g., seconds rather than minutes). This depolarization within a short amount of time may enable the sensor 12 to be used a short time after the defibrillation or similar procedure. Generally, any suitable conductive material may be used for the electrode 16 and the plurality of conductors 84. In other embodiments, instead of or in addition to including a portion printed on the backing layer 60, the electrode 16 and/or the plurality of conductors 84 are separate from the backing layer 60 and attached to the backing layer 60.

The plurality of conductors 84, as noted above, is configured to transmit signals to and/or from the electrode 16. In the illustrated embodiment, the backing layer 60 includes a tail portion 72 (FIGS. 2A-3) onto which the plurality of conductors 84 may be formed to or attached to extend from the electrode 16, for example, as a data and/or power connection and/or interface. The tail portion 72 may be a flat, flexible protrusion from the backing layer 60 to enable the sensor 12 to be worn by the patient with minimal discomfort by reducing the bulk and weight of the sensor 12 on the patient.

In some embodiments, the tail portion 72 and the plurality of conductors 84 may connect with connector 20, illustrated and described above, thereby providing an electrical and structural interface between the sensor 12 and the monitor 14 of FIG. 1. As an example, the connector 20 may be configured to enable the sensor 12 to clip into a connection point of monitor 14. The connector 20 may also include a memory unit configured to store information relating to the sensor 12, and to provide the stored information to the monitor 14. For example, the memory unit may store code configured to provide an indication to the monitor 14 as to the make/model of the sensor 12, the time-in-operation of the sensor 12, or the like. Alternatively or additionally, the memory unit may include code configured to perform a time-out function where the sensor 12 is deactivated after a predetermined number of connections, time-in-operation, or similar use-related metric. In some embodiments, the memory unit may also store patient-specific and/or sensor-specific information such as trend data collected by the electrode 16, calibration data related to the electrode 16 and/or the plurality of conductors 84, and the like. In other words, the memory unit may be configured to enable the sensor 12 to be used in conjunction with the monitor 14 for the collection of patient data.

Although a specific example monitor 14 and sensor 12 is described herein, in other embodiments, the sensor 12 can be any suitable sensor including an electrically conductive material comprising abrasive particles and configured to connect to any suitable patient monitor or other device via another suitable configuration.

The sensor 12 may be kept in electrical contact with a patient for the collection of physiological data or similar data. To this end, the sensor 12 includes an electrically conductive gel 110 configured to facilitate the transmission of electrical signals between the electrode 16 and the patient tissue. In some embodiments, the electrically conductive gel 110 includes a wet gel or a hydrogel that is compatible with the materials used for the electrode 16 and the plurality of conductors 84. For example, the electrically conductive gel 110 may be electrolytic and may include a salt (e.g., sodium chloride (NaCl) or potassium chloride (KCl)) having an ionic concentration suitable for conducting electrical signals between the patient and the electrode 16. In particular, the concentration of chloride ions in the conductive gel may be approximately 2% to approximately 10% by weight.

The electrically conductive gel 110 includes abrasive particles 112, which are configured to help improve the electrical contact between the electrode 16 and skin of a patient compared to embodiments in which the electrically conductive gel 110 does not include abrasive particles 112. In some embodiments, the abrasive particles 112 may be dispersed within the electrically conductive gel 110, submerged within the electrically conductive gel 110, or suspended or held in suspension within the electrically conductive gel 110. The abrasive particles 112 are configured to displace at least a portion of a layer, e.g., dead skin cells, body oils, dust, debris, or the like, on or of the surface of the skin of the patient and enable the electrically conductive gel 110 to contact and/or wet to the surface of the patient's skin. The abrasive particles 112 may comprise silica, aluminum oxide, or any suitable abrasive material compatible with use on skin. In some embodiments, the abrasive particles 112 each have a particles size ranging from a minimum dimension of 1 micrometer to a maximum dimension of 1 millimeter to ensure proper abrasion of the skin according to desired depth/intensity. In some embodiments, the abrasive particles 112 may be sharp, e.g., have one or more substantially sharp edges or points suitable for abrading skin, and may have a hardness suitable for abrading skin. In some embodiments, the abrasive particles 112 may be non-water-soluble. In some embodiments, the electrically conductive gel 110 may have a viscosity of between about 10,000 centipoise and about 100,000 centipoise.

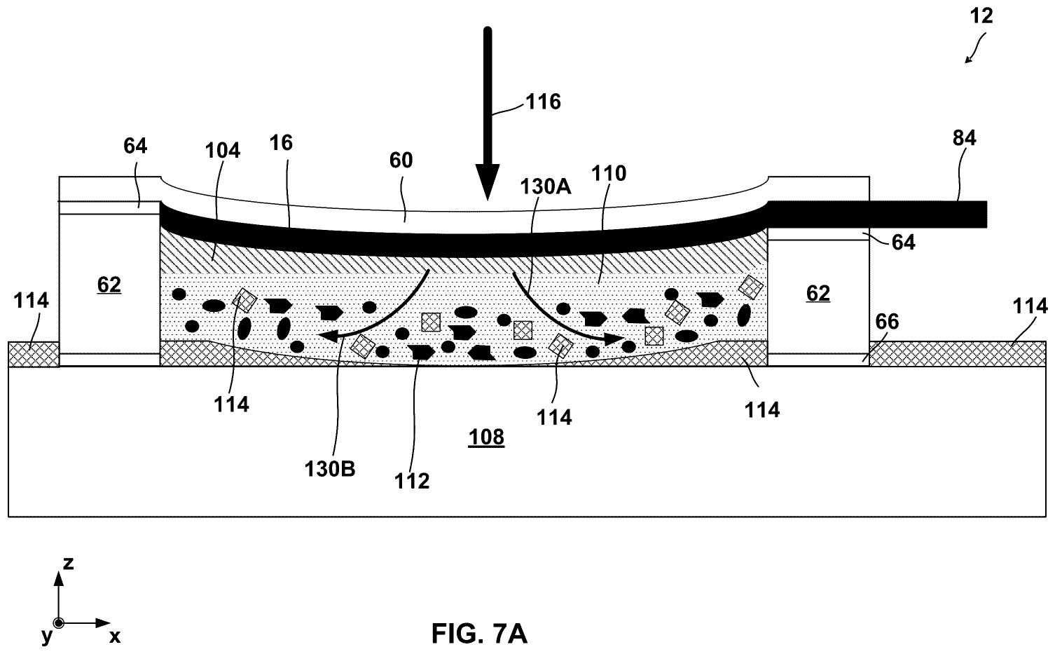

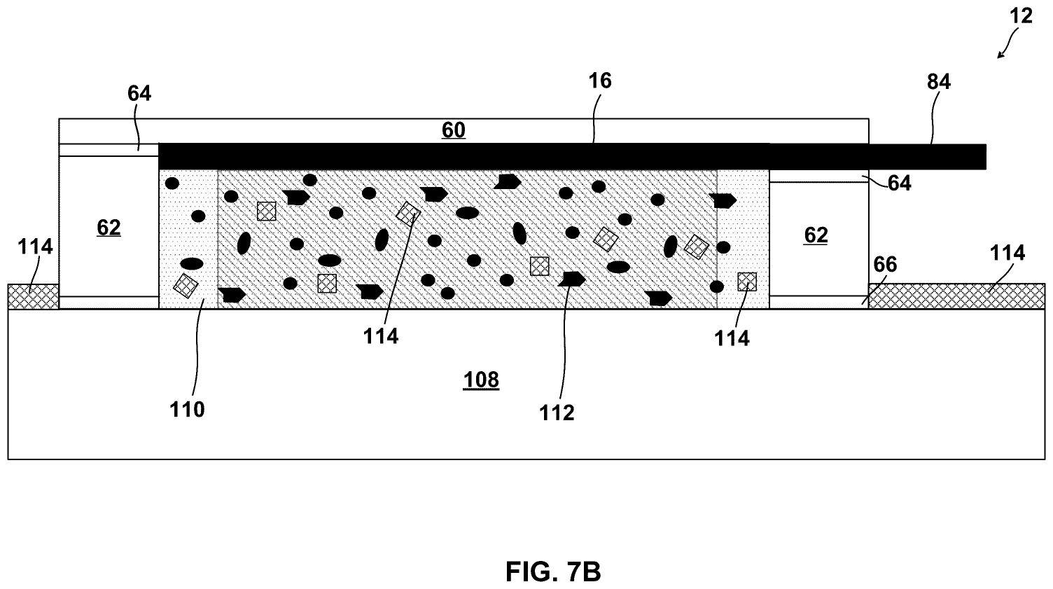

In some embodiments, prior to use of the sensor 12, the electrically conductive gel 110 may be retained in a retainer, such as a sponge 104, positioned within the electrode well 100. The sponge 104 may be disposed at least partially within the electrode well 100 when the electrode well 100 is uncovered, and may be compressible to fit within the electrode well 100 fully when covered, e.g., covered by either by the peelable layer 102 or the skin of the patient when the sensor 12 is applied. In other examples, the sponge 104 may be positioned to fully fit within the electrode well 100 when covered or uncovered. The sponge 104 is configured to release the electrically conductive material 110 and the abrasive particles 112 to at least partially fill the electrode well 100, e.g., upon the application of a force to the electrode well 100, which may compress the electrode well 100 and the sponge 104, or upon application of the sensor 12 to the patient, which may compress the sponge 104. In some embodiments, the conductive gel 110 and the abrasive particles 112 are configured to operate with the sponge 104. For example, the viscosity of the conductive gel 110 including the abrasive particles 112, and the sizes and density of the abrasive particles 112, are such that the conductive gel 110 and the abrasive particles 112 release from the sponge 104 and flow within the electrode well 100 to abrade layer 114 and/or skin 108 upon compression of the sponge 104 (FIG. 7A).

In other embodiments, the sensor 12 may not include a retainer, or the sponge 104. For example, the electrically conductive gel 110 (including the abrasive particles 112) may partially or fully fill the electrode well 100. In still other embodiments, the sensor 12 may include the sponge 104 and additional components, such as a spacer (not shown) configured to position the sponge 104 at a desired depth location within the electrode well 100 and/or create a gap between the sponge 104 and the electrode 16 to allow, or improve, contact or wetting between the electrically conductive gel 110 and the surface of the electrode 16.

In some embodiments, the sensor 12 may be part of an assembly. For example, the sensor 12 may include an electrode assembly including the electrode well 100 and the electrically conducting material 110 including the abrasive particles 112. The assembly may include the peelable layer 102 along with sensor 12, e.g., adhered to the patient contacting adhesive 66 and protecting the patient contacting adhesive 66 and containing the electrically conductive material 110 and abrasive particles 112 within electrode well 100 (e.g., during shipping and storing). The peelable layer 102 may be configured to contain the electrically conductive material 110 and the abrasive particles 112 within electrode well 100, e.g., by releasably adhering to, and covering, the patient contacting adhesive 66. In particular, the peelable layer 102 may be a tray, a liner, or the like, and may be configured to prevent the electrically conductive material 110 from drying out. When the sensor 12 is to be used, a user may remove and discard the peelable layer 102 and apply the sensor 12 to the patient.

In some embodiments, the sensor 12 does not include tines. For example, the sensor 12 may be configured with improve the conductivity between skin 108 of the patient (FIGS. 7A and 7B) and the electrode 16, or reduce an electrical impedance between the skin 108 of the patient and the electrode 16, without tines, and without a skin preparation step before applying the sensor 12.

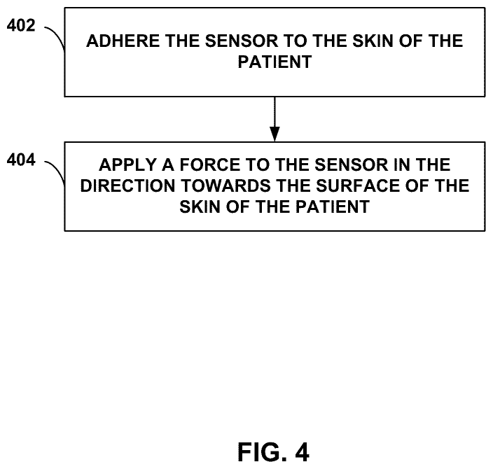

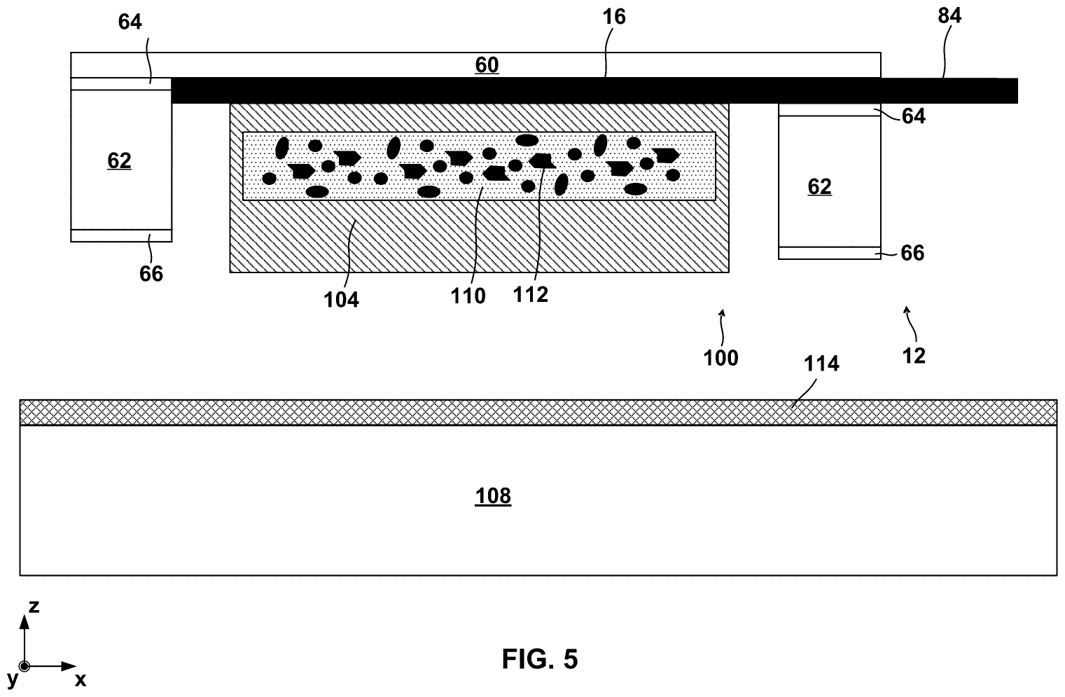

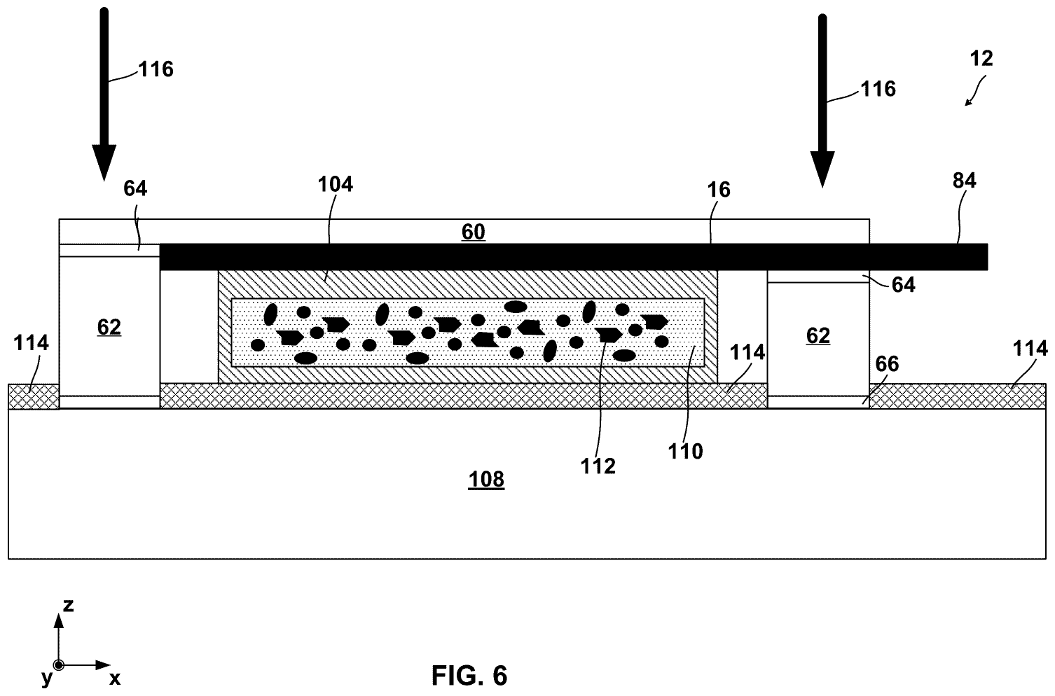

FIG. 4 is a flow diagram of an embodiments of a method of using the sensor 12 including the electrically conductive material 110 including the abrasive particles 112. While FIG. 4 is described with reference to the sensor 12 and the monitoring system 10, in other embodiments, the method can be used with other sensors and systems. FIG. 4 is described with reference to FIGS. 5-7B, which illustrate the sensor 12 at various steps of the method of FIG. 4. FIG. 5 is cross-sectional view of the sensor 12 of FIG. 2A before being applied to a patient, FIG. 6 is cross-sectional view of the sensor 12 of FIG. 2A applied to a patient and before a force is applied to the sensor 12 to press electrode well 100, FIG. 7A is a cross-sectional view of the sensor of FIG. 2A with a force applied to the sensor 12 to press the electrode well 100, and FIG. 7B is a cross-sectional view of the sensor of FIG. 2A applied to a patient and after the electrode well 100 is pressed, the cross-sections of FIGS. 5-7B being taken along the line A-A′ illustrated in FIG. 2A.

The user may adhere the sensor 12 to skin 108 of the patient (402). For example, the user may apply the sensor 12 to skin 108 by applying a force in direction 116 (FIG. 6) towards the skin 108 and about the perimeter of the electrode well 100 so as to adhere or otherwise affix the sensor 12 to the patient, e.g., via the patient contacting adhesive 66. Adhering the patient contacting adhesive 66 to the skin 108 may seal the electrode well 100, e.g., contain the electrically conductive material 110 and the abrasive particles 112 within the electrode well 100 (with skin 108 serving as a surface to close off the electrode well 100).

In some embodiments, prior to adhering the sensor 12 to the skin 108 of the patient, the user may remove the peelable layer 102 (e.g., a liner or release liner) from the patient contacting adhesive 66 to expose the patient contacting adhesive 66 and the electrode well 100, as shown in FIG. 5. Upon removal of the peelable layer 102, the sponge 104 may extend out of the electrode well 100 past the plane of the patient contacting adhesive 66, e.g., the sponge 104 may decompress, as shown in FIG. 5. In other embodiments, the sponge 104 may not extend out of the electrode well 100, and in still other embodiments, the sensor 12 may not include the sponge 104. The user may position the sensor 12 at or near skin 108 of the patient in a measurement area, e.g., such that the patient contacting adhesive 66 is closest to the skin 108. The user may then adhere the sensor 12 to the skin 108 of the patient at step (402).

In some embodiments, applying a force in the direction 116 to the sensor 12 to adhere the patient contacting adhesive 66 causes the abrasive particles 112 to displace at least a portion of layer 114 on, or of, the skin 108 of the patient and cause the electrically conductive material 110 to wet to the surface of the skin 108. For example, if the sponge 104 extends out of the electrode well 100 past the plane of the patient contacting adhesive 66 upon removal of the peelable layer 102, applying the force to adhere the patient contacting adhesive 66 may compress the sponge 104 and release at least a portion of the electrically conductive material 110 and the abrasive particles 112, causing the electrically conductive material 110 and the abrasive particles 112 to flow and/or move within the electrode well 100. The flow and/or movement of the electrically conductive material 110 and the abrasive particles 112 may then displace at least a portion of the layer 114. In other words, compressing the sponge 104 during application of the sensor 12 to the skin 108 may be a “one step” application and cleaning and/or preparation of the skin 108 to at least partially remove at least a portion of the layer 114 (e.g., dead skin cells, body oils, dust, debris, and the like) and increase a surface area of wetting of the electrically conductive material 110 directly to the skin 108 (e.g., directly to an outer surface of the epidermis of the patient) and decrease an impedance between the skin 108 and the electrode 16.

As illustrated in FIG. 7A, after the sensor 12 is adhered to the skin 108, the user may apply additional force to the sensor 12 in direction 116 towards the surface of the skin 108 (404). The application of the force to the sensor 12 in the direction 116 causes the abrasive particles 112 to displace at least a portion of the layer 114 on or of the skin 108 of the patient, and may cause the electrically conductive material 110 to wet to the surface of the skin 108. For example, after adhering the sensor 12 to the skin 108, the user may press and/or rub more directly on an area of the backing layer 60 corresponding to the electrode well 100, e.g., more towards a center portion of the sensor 12. The electrode well 100 maybe compressible, flexible, or the like, e.g., any or all of backing layer 60, the electrode 16, the first adhesive 64, the foam layer 62, and the patient contacting adhesive may be flexible, and the pressing or rubbing may compress and/or change the shape of the internal volume of the electrode well 100 to cause the electrically conductive material 110 and the abrasive particles 112 to flow and/or move within the electrode well 100, e.g., as indicated by flow directions 130A and 130B.

The electrode well 100 (e.g., the backing layer 60, the electrode 16, the first adhesive 64, the foam layer 62, and the patient contacting adhesive 66) contains and/or retains the electrically conductive material 110 and the abrasive particles 112, and the flow and/or movement of the abrasive particles 112 within the electrode well 100 displaces at least a portion of the layer 114 on the surface of the skin 108. For example, the force in direction 116 (the negative z-axis direction as shown) compresses the backing layer 60 and the flexible electrode 16, compressing the sponge 104 and causing the electrically conductive material 110 and the abrasive particles 112 to move (e.g., flow) towards the skin 108, e.g., in the z-axis direction and the negative and positive x-axis directions. In the example shown in FIG. 7A, the electrically conductive material 110 and the abrasive particles 112 move in flow directions 130A and 130B at least because the flow is constrained by the electrode well 100 and the skin 108. The compression causes the electrically conductive material 110 and the abrasive particles 112 to increase pressure against the skin 108 and the layer 114 (in the z-axis direction) and to flow along the skin 108 and the layer 114 (in the positive and negative x-axis directions). The compression-caused flow of the electrically conductive material 110 and the abrasive particles 112 causes the abrasive particles 112 to scrape the layer 114, e.g., as a mild abrasive, along the surface of the layer 114, displacing at least a portion of the layer 114 and exposing the skin 108, e.g., the epidermis and/or dermis layers of skin 108 underneath the layer 114.

In some embodiments, the sponge 104 may not extend out of the electrode well 100 upon removal of the peelable layer 102, and applying the force to the sensor 12 may cause the sponge 104 to release at least a portion of the electrically conductive material 110 and the abrasive particles 112, causing the electrically conductive material 110 and the abrasive particles 112 to flow and/or move within the electrode well 100. For example, as illustrated in FIG. 6, the electrically conductive material 110 and the abrasive particles 112 are retained within the sponge 104 after application of the sensor 12 to the skin 108 to adhere the sensor 12 to the skin 108. The user may then press and/or rub on the sensor 12 (e.g., an outer surface of the backing layer 60), and the resulting flow and/or movement of the electrically conductive material 110 and the abrasive particles 112, e.g., in flow directions 130A and 130B, may then displace at least a portion of the layer 114 as shown in FIG. 7A.

In some embodiments, the electrode well 100 may not include the sponge 104, and applying the force to the sensor 12 may cause the electrically conductive material 110 and the abrasive particles 112 to flow and/or move within the electrode well 100. The flow and/or movement of the electrically conductive material 110 and the abrasive particles 112 may then displace at least a portion of the layer 114 as described above. In embodiments in which the electrode well 100 includes the sponge 104 that extends out of the electrode well 100 upon removal of the peelable layer 102 and that is compressed during application of the sensor 12 to the skin 108, applying the force to the sensor 12 (e.g., pressing and/or rubbing an outer surface of the backing layer 60 that corresponds to the electrode well 100) may further compress the sponge 104 and cause the sponge 104 to further release an increased portion of the electrically conductive material 110 and the abrasive particles 112, and further cause the electrically conductive material 110 and the abrasive particles 112 to flow and/or move within the electrode well 100.

In some embodiments, the user may apply a force to the sensor 12 in the direction 116 towards the surface of the skin 108. In some embodiments, applying the force to adhere the sensor 12 (402) and to displace at least a portion of the layer 114 (e.g., “clean” skin 108) (404) may be a single step. For example, the user may press several areas of the sensor 12, or areas corresponding to the electrode well 100 or the patient contacting adhesive 66, which may both cause the patient contacting adhesive 66 to adhere to the skin 108 and the electrode well 100 to flex and the electrically conductive material 110 and the abrasive particles 112 to flow. In some embodiments, applying a force to the sensor 12 (404) may compress and/or depress the foam layer 62 in a direction towards the skin 108, and in some embodiments, depress the first adhesive 64 and the patient contacting adhesive 66 as well and may compress the sidewalls of the electrode well 100, which may be defined by the foam layer 62. In some embodiments, applying a force to the sensor 12 (404) may cause the electrically conductive material 110 and the abrasive particles 112 to flow to fill the volume of the electrode well 100.

In some embodiments, the electrode well 100 may retain the electrically conductive material 110 and the abrasive particles 112 after the user applies a force to the sensor 12, may reduce interference with the patient contacting adhesive 66 and the skin 108, e.g., which may improve adhesion between the patient contacting adhesive 66 and the skin 108. For example, the patient contacting adhesive 66, the foam layer 62, the first adhesive 64, and the backing layer 60 may form a barrier such that the electrically conductive material 110 and the abrasive particles 112 cannot escape or flow out of the electrode well 100. In contrast, a sensor utilizing a skin preparation step before applying the sensor 12 to the skin 108, such as an abrasive gel, may leave at least a residue and/or material between the patient contacting adhesive 66 and the skin 108, which may interfere with the adhesion of the sensor 12 to the skin 108.

In some embodiments, the electrically conductive material 110 may absorb at least a portion of the layer 114 after at least a portion of the layer 114 is displaced by the abrasive particles 112 (FIG. 7B). For example, when pressing or rubbing on the sensor 12 and causing the electrically conductive material 110 and the abrasive particles 112 to flow and displace at least a portion of the layer 114 from the surface of the skin 108, the electrically conductive material 110 may flow and absorb dead skin cells, body oils, dust, debris, and the like, and/or hold at least a portion of the layer 114, e.g., the dead skin cells, body oils, dust, debris, and the like, in suspension and away from the surface of the skin 108, and away from the surface of the electrode 16.

In some embodiments, the method of FIG. 4 may be used to improve the conductivity between the skin 108 of the patient and the electrode 16, and/or decrease or reduce an electrical impedance between the skin 108 of the patient and the electrode 16, without a skin preparation step before applying the sensor 12. For example, the user may adhere the sensor 12 to the patient (402) without pre-preparing the skin, e.g., without cleaning, abrading, sanding, or otherwise preparing the skin 108, and press on the sensor 12 (404) to cause the abrasive particles 112 to displace at least a portion of the layer 114 on, or of the skin 108 to reduce the impedance between the skin 108 of the patient and the electrode 16.

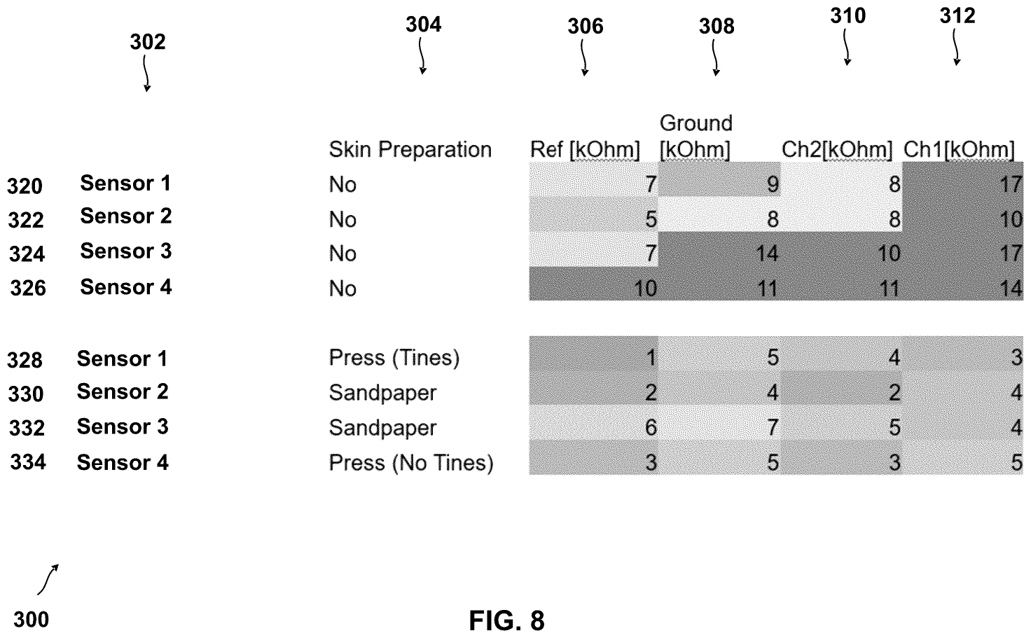

FIG. 8 is a table 300 illustrating a comparison of the impedances of several sensors and/or configurations of sensors. Table 300 includes columns 302-312 and rows 320-334. Column 302 indicates four example sensor types (rows 320-326, Sensors 1-4) without a skin preparation step and the same four sensor types (rows 328-334, Sensors 1-4) with a skin preparation step. The sensor types are a bispectral index sensor with an electrolytic gel and tines (row 320, Sensor 1), a sensor with a dry gel, e.g., a gel having a higher viscosity and/or low or no flow, which may be, for example, a dry gel used with a NIM-Eclipse® intraoperative nerve monitoring system from Medtronic, Inc. (row 322, Sensor 2), an ECG electrode sensor (row 324, Sensor 3), and a bispectral index sensor 12 with the electrically conductive material 110 and the abrasive particles 112 (row 326, Sensor 4). Column 304 indicates a skin preparation type, e.g., none for rows 320-326, press (e.g., applying a force to the sensor) for rows 328 and 334, and sandpaper (e.g., abrading the skin with sandpaper) for rows 330 and 332. Columns 306-312 indicate an impedance of different readout channels indicative of an impedance in kiloohms (kOhm) between the skin 108 and an electrode of the respective electrode for each corresponding row.

In the example shown, rows 320-326 for each of the four sensors without a skin preparation step indicate a significantly higher impedance between the skin 108 and a respective electrode. For example, rows 320 and 326 are BIS sensors adhered to the skin 108 but without pressing on the sensor, e.g., to cause the tines (row 320) or abrasive particles (row 326) to remove at least a portion of the layer 114. Rows 322 and 324 are sensors adhered to skin 108 without a skin pre-preparation step, e.g., abrading or rubbing the skin with sandpaper, a separate abrasive gel, or the like, before applying the respective sensor.

In the example shown, rows 328-334 for each of the four sensors with a skin preparation step indicate a significantly lower impedance between the skin 108 and a respective electrode. For example, rows 328 and 334 are BIS sensors adhered to the skin 108 and with, or after, pressing on the sensor, e.g., to cause the tines (row 320) or abrasive particles (row 326) to remove at least a portion of the layer 114. Rows 330 and 332 are sensors adhered to skin 108 with, or after, a skin pre-preparation step, e.g., abrading or rubbing the skin with sandpaper, a separate abrasive gel, or the like, before applying the respective sensor. In the example shown, rows 326 and 334 correspond to the sensor 12, and indicates a decrease in impedance between the skin 108 and the electrode 16 after application of the sensor 12 to the skin 108 and pressing on the sensor 12 to cause the abrasive particles 112 to displace at least a portion of the layer 114, where the sensor 12 is without, e.g., does not include, tines, and without a skin pre-preparation step, e.g., before application or adhering of the sensor 12 to the skin 108.

The following examples are a non-limiting list of examples in accordance with one or more techniques of this disclosure.

Example 1: A sensor including: an electrode assembly including an electrode well; and an electrically conductive material disposed within the electrode well and comprising abrasive particles, wherein the abrasive particles are configured to displace at least a portion of a layer on or of skin of a patient upon application of the sensor to the skin of the patient.

Example 2: The sensor of example 1, wherein the electrically conductive material is configured to wet to the surface of the skin and to wet to a surface of an electrode within the electrode well.

Example 3: The sensor of example 2, wherein the abrasive particles are configured to displace the at least a portion of the layer on or of the skin to layer increase a surface area of wetting between the electrically conductive material and the skin of the patient.

Example 4: The sensor of examples 2 or example 3, wherein the abrasive particles are configured to displace at least a portion of an outermost layer of the skin to decrease an impedance between the skin and the electrode.

Example 5: The sensor of any one of examples 1 through 4, wherein the electrically conductive material comprises an electrolytic gel.

Example 6: The sensor of any one of examples 1 through 5, wherein the electrically conductive material is configured to absorb the at least a portion of the layer on or of the skin after the at least the portion of the layer is displaced by the abrasive particles.

Example 7: The sensor of any one of examples 1 through 6, wherein the electrode assembly includes: a backing layer; at least one electrode disposed on the backing layer; a foam layer disposed on at least a portion of the backing layer; and an adhesive disposed on at least a portion of the foam layer and configured to adhere the sensor to a patient, wherein the electrode well is defined by the foam layer and the backing layer.

Example 8: The sensor of any one of examples 1 through 7, further includes a sponge disposed at least partially within the electrode well and configured to release the electrically conductive material upon application of the sensor to the skin of the patient.

Example 9: The sensor of example 7 or example 8, wherein the adhesive, foam layer, and backing layer are configured to contain the electrically conductive material.

Example 10: The sensor of any one of examples 1 through 9, wherein the electrode well is deformable upon the application of a pressure to the electrode assembly to cause the abrasive particles to displace the at least the portion of the layer on or of the skin, wherein the electrode well is configured to contain the electrically conductive material after being deformed in response to the application of the pressure.

Example 11: The sensor of any one of examples 1 through 10, wherein the sensor is at least one of an electroencephalogram sensor or an electrocardiogram sensor.

Example 12: An assembly including: a sensor including: an electrode assembly including an electrode well, the electrode assembly including: a backing layer; an electrode disposed on the backing layer; a foam layer disposed on at least a portion of the backing layer; an adhesive disposed on at least a portion of the foam layer and configured to adhere the sensor to a patient, wherein the electrode well is defined by the foam layer and the backing layer; and an electrically conductive material disposed within the electrode well and comprising abrasive particles, wherein the abrasive particles are configured to displace at least a portion of a layer on or of a skin of a patient upon application of the sensor to the skin of the patient; and a peelable layer configured to contain the electrically conductive material within the electrode well and configured to releasably adhere to, and cover, an outer surface of the adhesive.

Example 13: The assembly of example 12, wherein the electrically conductive material is configured to wet to the surface of the skin and to wet to a surface of an electrode within the electrode well.

Example 14: The assembly of example 13, wherein the abrasive particles are configured to displace at least a portion of the layer on the surface of the skin to layer increase a surface area of wetting between the electrically conductive material and the skin of the patient.

Example 15: The assembly of example 13 or example 14, wherein the abrasive particles are configured to displace at least a portion of an outermost layer of the skin to decrease an impedance between the skin and the electrode.

Example 16: The assembly of any one of examples 12 through 15, wherein the electrically conductive material comprises an electrolytic gel.

Example 17: The assembly of any one of examples 12 through 16, wherein the electrically conductive material is configured to absorb the at least a portion of the layer on or of the skin after the at least the portion of the layer is displaced by the abrasive particles.

Example 18: The assembly of any one of examples 12 through 17, further includes a sponge disposed at least partially within the electrode well and configured to release the electrically conductive material.

Example 19: The assembly of any one of examples 12 through 18, wherein the adhesive, foam layer, and backing layer are configured to contain the electrically conductive material after application of the sensor to the skin of the patient.

Example 20: The assembly of any one of examples 12 through 19, wherein the electrode well is deformable upon the application of a pressure to the electrode assembly to cause the abrasive particles to displace the at least the portion of the layer on or of the skin, wherein the electrode well is configured to contain the electrically conductive material after being deformed in response to the application of the pressure.

Example 21: The assembly of any one of examples 12 through 20, wherein the sensor is at least one of an electroencephalogram sensor or an electrocardiogram sensor.

Example 22: A method including: positioning a sensor on a surface of a skin of a patient, the sensor including: an electrode assembly including an electrode well; and an electrically conductive material including abrasive particles; applying a force to the sensor to adhere the adhesive to the surface about a perimeter of the electrode well thereby sealing the electrode well; and applying a force to the sensor in a direction towards the surface of the skin, wherein the application of the force causes the abrasive particles to displace at least a portion of a layer on or of the skin of the patient

Example 23: The method of example 22, further comprising applying a force to the sensor to adhere an adhesive to the surface about the perimeter of the electrode well thereby sealing the electrode well.

Example 24: The method of example 22 or example 23, wherein the at least the portion of the layer on or of the skin includes an outermost layer of an epidermis of the patient.

Example 25: The method of any of examples 22 through 24 or any of examples 22 through 24, wherein the electrically conductive material is configured to wet to the surface of the skin and to wet to a surface of an electrode within the electrode well.

Example 26: The method of example 25, wherein displacing the at least the portion of the layer on or of the skin increases a surface area of wetting between the electrically conductive material and the skin of the patient.

Example 27: The method of any one of examples 24 through example 26, wherein displacing the at least the portion of the layer on or of the skin of the patient decreases an impedance between the skin and the electrode.

Example 28: The method of any one of examples 22 through 27, wherein the electrically conductive material comprises an electrolytic gel.

Example 29: The method of any one of examples 22 through 28, wherein the electrode assembly includes: a backing layer; at least one electrode disposed on the backing layer; a foam layer disposed on at least a portion of the backing layer; a sponge disposed at least partially within the electrode well and configured to retain the electrically conductive material upon application of the sensor to the skin of the patient; and an adhesive disposed on at least a portion of the foam layer and configured to adhere the sensor to a patient, wherein the electrode well is defined by the foam layer and the backing layer, wherein applying the force to the sensor causes the sponge to compress to release at least a portion of the electrically conductive material into the electrode well.

Example 30: The method of example 29, wherein after applying the force to the sensor, the electrically conductive material is retained within the electrode well by the adhesive, the foam layer, and backing layer.

Example 31: The method of any one of examples 22 through 30, wherein the sensor is at least one of an electroencephalogram sensor or an electrocardiogram sensor.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device. These and other examples are within the scope of the following claims.

What is claimed is:

1. A sensor, comprising:
an electrode assembly including an electrode well; and
an electrically conductive material disposed within the electrode well and comprising abrasive particles, wherein the abrasive particles are configured to displace at least a portion of a layer on or of skin of a patient upon application of the sensor to the skin of the patient.

2. The sensor of claim 1, wherein the electrically conductive material is configured to wet to a surface of the skin and to wet to a surface of an electrode within the electrode well.

3. The sensor of claim 2, wherein the abrasive particles are configured to displace the at least a portion of the layer on the surface of the skin to increase a surface area of wetting between the electrically conductive material and the skin of the patient.

4. The sensor of claim 1, wherein the abrasive particles are configured to displace at least a portion of an outermost layer of the skin to decrease an impedance between the skin and the electrode.

5. The sensor of claim 1, wherein the electrically conductive material comprises an electrolytic gel.

6. The sensor of claim 1, wherein the electrically conductive material is configured to absorb the at least a portion of the layer after the at least the portion of the layer is displaced by the abrasive particles.

7. The sensor of claim 1, wherein the electrode assembly comprises:
a backing layer;
at least one electrode disposed on the backing layer;
a foam layer disposed on at least a portion of the backing layer; and
an adhesive disposed on at least a portion of the foam layer and configured to adhere the sensor to the patient, wherein the electrode well is defined by the foam layer and the backing layer.

8. The sensor of claim 7, further comprising:
a sponge disposed at least partially within the electrode well and configured to release the electrically conductive material upon application of the sensor to the skin of the patient.

9. The sensor of claim 7, wherein the adhesive, the foam layer, and the backing layer are configured to contain the electrically conductive material upon application of the sensor to the skin of the patient.

10. The sensor of claim 1, wherein the electrode well is deformable upon application of a force to the electrode assembly causing the abrasive particles to displace the at least a portion of the layer on or of the skin, wherein the electrode well is configured to contain the electrically conductive material after being deformed in response to the application of the force.

11. The sensor of claim 1, wherein the sensor is at least one of an electroencephalogram sensor or an electrocardiogram sensor.

12. An assembly comprising:
a sensor comprising:
an electrode assembly including an electrode well, the electrode assembly comprising:
a backing layer;
an electrode disposed on the backing layer;
a foam layer disposed on at least a portion of the backing layer;
an adhesive disposed on at least a portion of the foam layer and configured to adhere the sensor to a patient, wherein the electrode well is defined by the foam layer and the backing layer; and
an electrically conductive material disposed within the electrode well and comprising abrasive particles, wherein the abrasive particles are configured to displace at least a portion of a layer on or of a skin of the patient upon application of the sensor to the skin of the patient; and
a peelable layer configured to contain the electrically conductive material within the electrode well and configured to releasably adhere to and cover an outer surface of the adhesive.

13. The assembly of claim 12, wherein the electrically conductive material is configured to wet to a surface of the skin and to wet to a surface of an electrode within the electrode well.

14. The assembly of claim 13, wherein the abrasive particles are configured to displace the at least a portion of the layer on the surface of the skin to increase a surface area of wetting between the electrically conductive material and the skin of the patient.

15. A method, comprising:
positioning a sensor on a surface of a skin of a patient, the sensor comprising:
an electrode assembly including an electrode well; and
an electrically conductive material comprising abrasive particles;
applying a force to the sensor to adhere the sensor to the surface about a perimeter of the electrode well thereby sealing the electrode well; and
applying a force to the sensor in a direction towards the surface of the skin, wherein the application of the force causes the abrasive particles to displace at least a portion of a layer on the surface of the skin of the patient.
